(19) United States
(12) Patent Application Publication
(10) Pub. No.: US 2021/0068759 A1
(43) Pub. Date: Mar. 11, 2021

(54) BIO-FEEDBACK CUP WITH BIO-SIGNAL ACQUISITION AND FEEDBACK CAPABILITY

(71) Applicant: HAOSEN TECH CO., LTD., Taoyuan City (TW)

(72) Inventor: KUO-YUAN CHUNG, New Taipei City (TW)

(21) Appl. No.: 16/565,508

(22) Filed: Sep. 10, 2019

(51) Int. Cl. A61B 5/00; A61B 5/0205; A61B 5/01; A61B 5/0402; A61B 5/1455; A61B 5/16

(52) U.S. Cl. CPC A61B 5/6887; A61B 5/02055; A61B 5/01; A61B 5/0402; A61B 5/486; A61B 5/14551; A61B 5/02427; A61B 5/165; A61B 5/742; A61B 5/0006; A61B 5/0008; A61B 5/0017; A61B 5/7275

(57) ABSTRACT

A bio-feedback cup with bio-signal acquisition and feedback includes a cup body, a grip, a first electrode, a second electrode, a human-body temperature sensor module, a photoplethysmography (PPG) sensor module, a bio-signal acquisition module, and a bio-feedback module. The bio-signal acquisition module is electrically connected to the first electrode, the second electrode, a ground electrode, the human-body temperature sensor module and the PPG sensor module, and is configured to detect the user's cardiac signals with a built-in electrocardiogram (ECG or EKG) acquisition module. The bio-feedback module is electrically connected to the bio-signal acquisition module and is configured to instantly give the user corresponding bio-feedback by changing a pattern on a surface of the cup body, according to a physiological index and an extended cardiac index of the user.

### FIELD OF THE DISCLOSURE

The present disclosure relates to a cup, and more particularly to a bio-feedback cup with bio-signal acquisition and feedback capability.

### BACKGROUND OF THE DISCLOSURE

In recent years, since the importance of health has been emphasized, people wear various smart wearable devices, such as wristwatches or wristbands, to measure their daily bio-signals, and those devices serve as an important interface for tracking of bio-signals and cardio-health management. However, for people who don't wear wristwatches in their daily lives, wearing such devices becomes a burden, forcing them to adapt to unfamiliar materials or giving up their favorite clothing coordination. Therefore, if the measurement interface of the bio-signals can be an item people use every day, it will not cause any additional burden on people, or force them to change their habits or clothing coordination. Measurement behavior is always considered repetitive, boring, and not directly beneficial, so that people often lose the motivation to continuously conduct measurements and long-term tracking. Therefore, incorporating such tedious measurements into people's daily lives has always been an issue to be solved. The present disclosure provides a cup that can easily achieve daily psychological and physical health management.

### SUMMARY OF THE DISCLOSURE

One of the objectives of the present disclosure is to provide a bio-feedback cup with bio-signal acquisition and feedback capability that can overcome the aforementioned drawbacks.

In one aspect, the present disclosure provides a bio-feedback cup with bio-signal acquisition and feedback capability, including a cup body, a grip, a first electrode, a second electrode, a human-body temperature sensor module, a photoplethysmography (PPG) sensor module, a bio-signal acquisition module, and a bio-feedback module. The grip is connected to the cup body. The first electrode is located at one of the grip or the cup body. The second electrode is located at the cup body. The human-body temperature sensor module is located at one of the grip or the cup body. The PPG sensor module is located at one of the grip or the cup body. The bio-signal acquisition module is electrically connected to the first electrode, the second electrode, a ground electrode, the human-body temperature sensor module and the PPG sensor module, and is configured to detect the user's cardiac signals with a built-in electrocardiogram (ECG or EKG) acquisition module. The bio-signal acquisition module detects and obtains an ECG signal when the user touches the first electrode and the second electrode with both hands. The bio-signal acquisition module obtains a PPG signal through the PPG sensor module when the user touches the PPG sensor module. The bio-signal acquisition module obtains a human-body temperature signal through the human-body temperature sensor module when the user touches the human-body temperature sensor module. The bio-feedback module is electrically connected to the bio-signal acquisition module and is configured to instantly give the user corresponding bio-feedback by changing a pattern on a surface of the cup body, according to a physiological index and an extended cardiac index of the user.

Preferably, the physiological index that can be used as instant feedback to the user and can be recorded, includes any one or more of human-body temperature, heart rate (beats per minute, BPM), heart rate variability (HRV), peripheral oxygen saturation (SpO2), heart age, blood pressure estimates and risk alert for excessive heart rate.

Preferably, the extended cardiac index that can be determined by a preset algorithm according to physiological data, ECG data and PPG data of the user, is a mood index, a stress index, a caffeine intake index or a heart risk index. Preferably, the first electrode, the second electrode and the ground electrode each is a thin metal plating layer, a thin metal coating layer, a thin metal sputter coating layer or a sheet-metal layer on the surface of the cup body or the grip.

Preferably, the bio-feedback module is configured to instantly give the user corresponding bio-feedback by changing a color, a shape, a text, a carving or a background illuminating color of the pattern on the surface of the cup body, according to the physiological index and the extended cardiac index.

Preferably, the bio-feedback module is configured to instantly give the user corresponding bio-feedback by further changing a color and a displayed length of a quantifiable color indicator exposed from the cup body, according to the physiological index and the extended cardiac index.

Preferably, the bio-feedback cup with bio-signal acquisition and feedback capability includes a wireless data transmission module and a wireless data transmission antenna. The wireless data transmission module and the wireless data transmission antenna are electrically connected to the bio-signal acquisition module for receiving and utilizing any one of Bluetooth, Wi-Fi and infrared (IR) wireless transmission technologies to transmit the user's physiological data to an electronic device.

Preferably, the wireless data transmission antenna is a thin metal plating layer, a thin metal coating layer, a thin metal sputter coating layer or a sheet-metal layer on the surface of the cup body or the grip.

Preferably, the electronic device is a mobile phone, a wearable device, a laptop or desktop computer, or a tablet computer.

Preferably, physiological data of the user is accumulated to form a historical database, stored in the electronic device, and presented by a software application of the electronic device that serves as a platform for cardio-health management and an interface for interacting with the user.

Preferably, the bio-feedback cup with bio-signal acquisition and feedback capability includes a power module. The power module is electrically connected to the bio-signal acquisition module and is configured to provide electric power.

Preferably, the power module is a replaceable battery module, a rechargeable battery module, a detachable battery module, or a wireless rechargeable battery module.

These and other aspects of the present disclosure will become apparent from the following description of the embodiment taken in conjunction with the following drawings and their captions, although variations and modifications therein may be affected without departing from the spirit and scope of the novel concepts of the disclosure.

### BRIEF DESCRIPTION OF THE DRAWINGS

The present disclosure will become more fully understood from the following detailed description and accompanying drawings.

FIG. 1 is a perspective view of a cup of the present disclosure.

FIG. 2 is a partial exploded view of a cup of the present disclosure.

FIG. 3 is a schematic view of a cup of the present disclosure in operation.

FIG. 4 is a functional block diagram of a cup of the present disclosure.

FIG. 5 is a perspective view of another cup of the present disclosure.

### DETAILED DESCRIPTION OF THE EXEMPLARY EMBODIMENTS

The present disclosure is more particularly described in the following examples that are intended as illustrative only since numerous modifications and variations therein will be apparent to those skilled in the art. Like numbers in the drawings indicate like components throughout the views. As used in the description herein and throughout the claims that follow, unless the context clearly dictates otherwise, the meaning of “a”, “an”, and “the” includes plural reference, and the meaning of “in” includes “in” and “on”. Titles or subtitles can be used herein for the convenience of a reader, which shall have no influence on the scope of the present disclosure.

The terms used herein generally have their ordinary meanings in the art. In the case of conflict, the present document, including any definitions given herein, will prevail. The same thing can be expressed in more than one way. Alternative language and synonyms can be used for any term(s) discussed herein, and no special significance is to be placed upon whether a term is elaborated or discussed herein. A recital of one or more synonyms does not exclude the use of other synonyms. The use of examples anywhere in this specification including examples of any terms is illustrative only, and in no way limits the scope and meaning of the present disclosure or of any exemplified term. Likewise, the present disclosure is not limited to various embodiments given herein. Numbering terms such as “first”, “second” or “third” can be used to describe various components, signals or the like, which are for distinguishing one component/signal from another one only, and are not intended to, nor should be construed to impose any substantive limitations on the components, signals or the like.

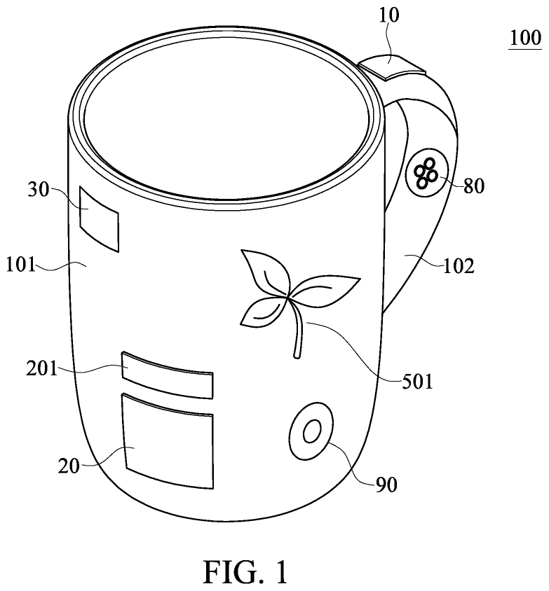

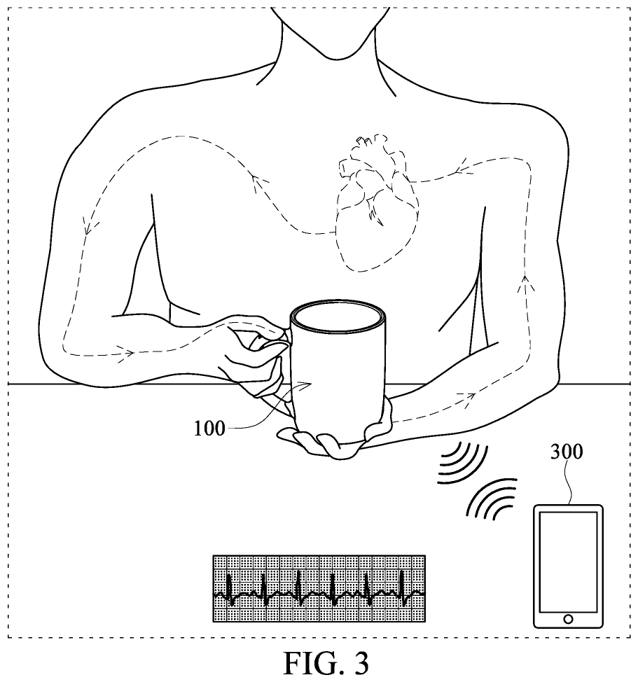

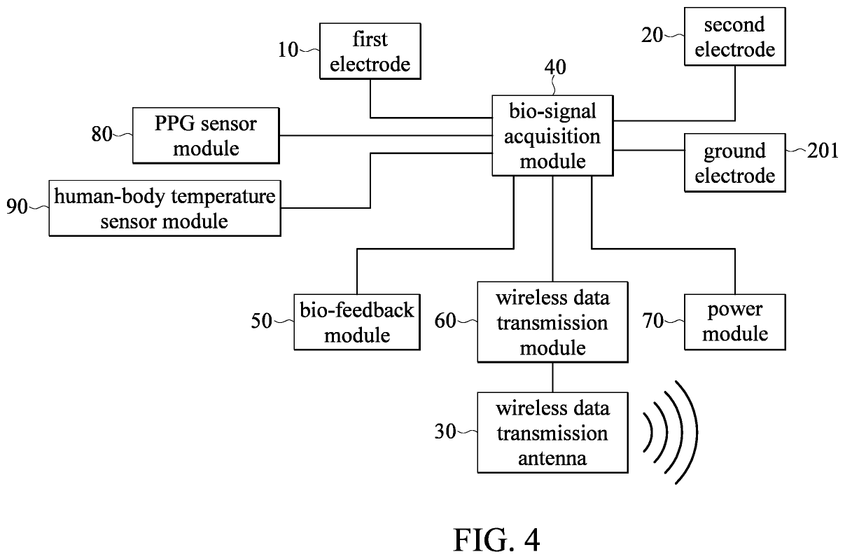

Referring to FIG. 1 to FIG. 4, the present disclosure provides a bio-feedback cup with bio-signal acquisition and feedback capability 100.

The bio-feedback cup with bio-signal acquisition and feedback capability 100 (hereinafter referred to as the cup 100) basically includes a cup body 101, a grip 102, a first electrode 10, a second electrode 20, a ground electrode 201, a wireless data transmission antenna 30, a bio-signal acquisition module 40, and a bio-feedback module 50. It is also possible that the cup 100 has no grip 102.

The bio-signal acquisition module 40 and the bio-feedback module 50 may be located at appropriate positions of the cup body 101 of the cup 100, or may be built-in as parts of the cup body 101. The shape of the cup body 101 can be adjusted according to practical requirements, and it can be a container without a grip, for which there is no restriction. Furthermore, in addition to the above-described modules, the cup 100 of the present embodiment may further include a wireless data transmission module 60 and a power module 70. The wireless data transmission antenna 30, the wireless data transmission module 60 and the power module 70 can be located at appropriate positions of the cup body 101 of the cup 100, or may be built-in as parts of the cup body 101. In addition, the cup 100 of the present embodiment may further include a PPG sensor module 80 and a human-body temperature sensor module 90.

The specific details of the embodiment, as well as the interaction relationships between various modules or elements, are further explained with examples below. It shall be specifically emphasized that the detailed characteristics described below are only for the convenience of those skilled in the art to more easily understand the contents of the present disclosure, and the specific implementation methods are not limited thereto.

The cup body 101 can be a liquid container integrally formed or assembled into a suitable shape that is made of a variety of materials, such as ceramics, stainless steel, and plastics. The grip 102 can be a hand-held part that is made of a variety of materials, and may be assembled on and detached from the cup body 101. The grip 102 can also be in the form of a cup sleeve or a cup holder for the cup body 101, so as to achieve the purpose of a grip for holding the cup. The first electrode 10 is located at the grip 102 connected to the cup body 101, and it can also be placed directly on the cup body 101 without the grip 102. The first electrode 10 can be a positive electrode made of a conductive material. The first electrode 10 may be made of at least one of the followings: a metal sheet, a conductive silicone/film, a thin metal plating layer on the surface of the cup body 101 or the grip 102, a thin metal coating layer, a thin metal sputter coating layer, or a thin metal plating layer. Its material and shape can be adjusted according to practical requirements or the shape of the grip 102. The first electrode 10 can be electrically connected to the bio-signal acquisition module 40 through conductive paths such as metal wires, films, or coating.

The second electrode 20 is located at the side edge of the cup body 101, and can be a negative electrode made of a conductive material. The second electrode 20 may be made of at least one of the following: a metal sheet, a conductive silicone/film, a thin metal plating layer on the surface of the cup body 101 or the grip 102, a thin metal coating layer, a thin metal sputter coating layer, or a thin metal plating layer. The material and shape of the second electrode 20 can be adjusted according to practical requirements or the shape of the cup body 101. The second electrode 20 can be electrically connected to the bio-signal acquisition module 40 through conductive paths such as metal wires, films, or coating.

The ground electrode 201 is located at the grip 102 connected to cup body 101, and may also be placed on the side edge of the cup body 101, which is not limited thereto. In some embodiments, a ground electrode may be omitted. The ground electrode 201 is made of a conductive material, such as a metal sheet, a conductive silicone, a film, a coating, and the material and shape thereof may be appropriately adjusted according to practical requirements. The ground electrode 201 can be electrically connected to the bio-signal acquisition module 40 through conductive paths such as metal wires, films, or coating.

The bio-signal acquisition module 40 can be located at the bottom of the cup body 101 and electrically connected to the first electrode 10, the second electrode 20 and the ground electrode 201, respectively. The bio-signal acquisition module 40 detects and obtains the cardiac signals of the user through the first electrode 10, the second electrode 20 and the ground electrode 201. In one embodiment, the bio-signal acquisition module 40 is or has an electrocardiogram (ECG or EKG) sensing circuit or integrated circuit. When the user touches the first electrode 10 and the second electrode 20 with both hands, in cooperation with the ground electrode 201, the body (heart) and the bio-signal acquisition module 40 cooperatively form a closed-loop circuit, thereby allowing the bio-signal acquisition module 40 to detect the user's cardiac signals and instantly record the current measurement to generate an electrocardiogram. In another embodiment, the bio-signal acquisition module 40 can receive electrical signals of the PPG sensor module 80. When the user's hand touches the PPG sensor module 80, the photoplethysmography (PPG) signals of the flowing substances in the blood can be detected during the pulses of the blood vessels, thereby calculating various physiological data (physiological index) such as the heart rate or various physiological data related to the blood.

In detail, electrocardiogram (ECG or EKG) sensing is to sense cardiac signals of the user. That is, every time the heart beats and the myocardial cells are depolarized, a small electrical change can be detected on the surface of the limb skins through the standard limb lead (Lead I) design. This small change can be captured and amplified by the ECG sensing circuit or the integrated circuit, and then the electrocardiogram can be drawn and recorded, which is well known to those skilled in the art and therefore will not be further described. Since the signals are weak and can be easily interfered, the integrated circuits of the amplifying circuit and the filtering circuit can be integrated into the bio-signal acquisition module 40 to amplify the weak signals and reduce the noise of the signals, so as to ensure the accuracy of detection and recording.

The bio-feedback module 50 is or has a pattern 501, which can be exposed from the cup body 101, and various physiological data of the user can be displayed through the shape or color changes of the pattern 501. When the user holds the grip 102 with one hand and touches the first electrode 10, and touches the cup body 101 with the other hand while touching the second electrode 20 at the same time, the bio-signal acquisition module 40 can detect and obtain the cardiac signals of the user through ECG and PPG sensor/module to record and calculate various physiological data thereof, such as the heart rate, heart rate variability, and so on, so that the bio-feedback module 50 gives the user corresponding bio-feedback according to various physiological data. For example, the bio-feedback module 50 can instantly give the user corresponding bio-feedback by changing the color of the cup body 101, or the color, shape or the background illuminating color of the pattern 501.

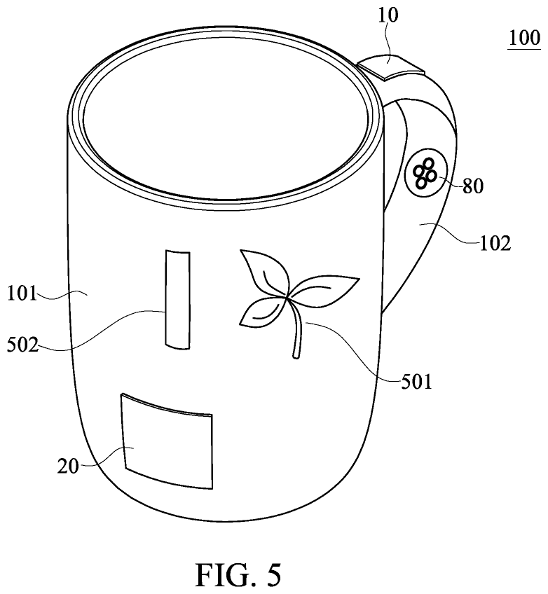

In one embodiment, as shown in FIG. 5, the bio-feedback module 50 may also be or have a quantifiable color indicator 502. The quantifiable color indicator 502 is exposed from the cup body 101 and used to provide bio-feedback according to various physiological data of the user, such as the heart rate, through the length and color changes. With the quantifiable color indicator 502, the user can more intuitively understand his/her cardiac physiological state, and it thereby provides the user with reference index value such as the caffeine intake.

In one embodiment, the cup 100 may have an expandable PPG sensor module 80, such as a photoplethysmography (PPG) sensor. Meanwhile, the signal output by the PPG sensor module 80 is a photoplethysmography (PPG) signal.

In detail, the PPG sensor module 80 uses the optical penetration and reflection methods and the principle of light sensing element absorbing light energy to detect changes in the amount of light absorbed and reflected by substances flowing through the blood vessels and blood when the peripheral blood vessel (such as small arteries) are pulsating. Further, since the blood flow in the blood vessels would change periodically according to heartbeat, and the PPG sensor module 80 senses the flow or concentration changes of the blood vessel tissue or blood content using the intensity of reflected light of the blood by illuminating the blood vessels under the skin with light of different wave lengths. At the same time, the period of the PPG signal is also corresponding to the period of heartbeat. That is, the PPG signal is the electrical signal generated in response to the flow or concentration in the blood vessel tissue or blood detected by the PPG sensor module 80 using the light sensing element. This is well known to those skilled in the art and therefore will not be further described. In the present embodiment, the PPG sensor module 80 may be exposed from or built-in on the cup body 101 or the grip 102, and electrically connected to the bio-signal acquisition module 40. When the user's finger touches the PPG sensor module 80, the bio-signal acquisition module 40 can calculate physiological data of the user, such as blood oxygen saturation, as a reference for health management according to the physiological data obtained by the PPG sensor module 80. Moreover, with both the PPG signals and the ECG signals, more physiological data of the user can be estimated, such as relative blood pressure estimates.

In one embodiment, the cup 100 may have an expandable human-body temperature sensor module 90, which may be an electronic thermometer or an infrared thermometer. The human-body temperature sensor module 90 is electrically connected to the bio-signal acquisition module 40 to obtain body temperature signals when contacting the user's hands. The human-body temperature can be calculated and recorded by the bio-signal acquisition module 40 based on the electrical signal obtained by the human-body temperature sensor module 90 when the user's finger touches the human-body temperature sensor module 90.

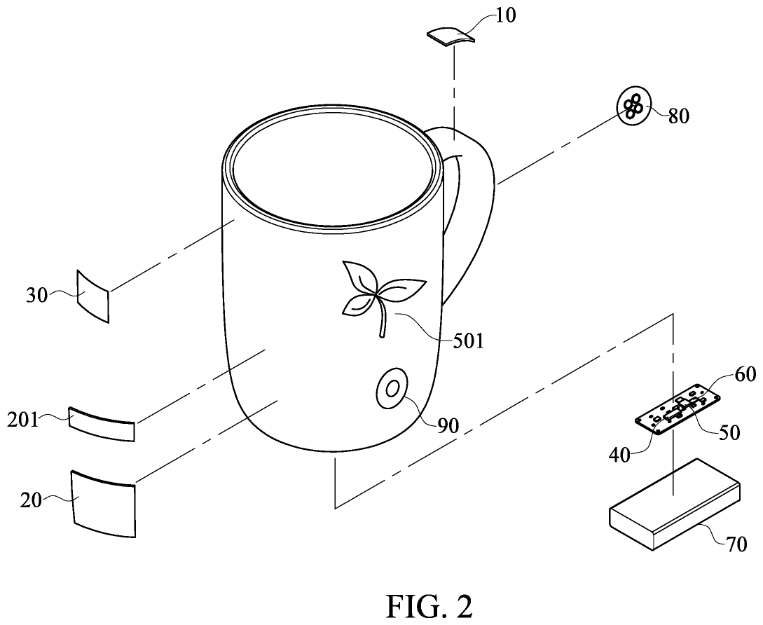

The wireless data transmission module 60 can be placed at the bottom of the cup body 101 as shown in FIG. 2 and electrically connected to the wireless data transmission antenna 30 and the bio-signal acquisition module 40. The bio-signal acquisition module 40 can send the detected raw physiological data or physiological information of the user to the bio-feedback module 50 or to the wireless data transmission module 60. The wireless data transmission module 60 sends the user's real-time physiological data through a wireless transmission manner such as Bluetooth, Wi-Fi or IR transmission technology to the electronic device 300, such as a mobile phone, a wearable device, a laptop or desktop computer, or a tablet computer, through the wireless data transmission antenna 30. After combining various algorithms on the bio-signal acquisition module 40 or the electronic device 300, various application information, such as human-body temperature, heart rate, heart rate variability, heart age, peripheral oxygen saturation, relative blood pressure, risk alert for excessive heart rate, mood index, physiological stress index, caffeine intake index, and heart risk index can be calculated.

In addition, the wireless data transmission antenna 30 may be at least one of the following: a thin metal plating layer, a thin metal coating layer, a thin metal sputter coating layer or a sheet-metal layer on the surface of the cup body 101 or the grip 102.

The power module 70 can be located at the bottom of the cup body 101 as shown in FIG. 2 and electrically connected to the bio-signal acquisition module 40 to provide the basic electric power of each module.

In summary, the exemplary embodiments of the present disclosure provide a bio-feedback cup with bio-signal acquisition and feedback capability. Through the first electrode located at the grip or the cup body, the second electrode located at the cup body, the PPG Sensor module located at the cup body or the grip, the human-body temperature sensor module located at the cup body or the grip, the bio-signal acquisition module designed with ECG detecting function located inside the cup body, and the bio-feedback module, the user can complete the measurement of the bio-signals while holding the cup without any additional burden on clothing coordination or being forced to change habits, and immediately obtain the corresponding bio-feedback.

The foregoing description of the exemplary embodiments of the disclosure has been presented only for the purposes of illustration and description and is not intended to be exhaustive or to limit the disclosure to the precise forms disclosed. Many modifications and variations are possible in light of the above teaching.

The embodiments were chosen and described in order to explain the principles of the disclosure and their practical application so as to enable others skilled in the art to utilize the disclosure and various embodiments and with various modifications as are suited to the particular use contemplated. Alternative embodiments will become apparent to those skilled in the art to which the present disclosure pertains without departing from its spirit and scope.

What is claimed is:

1. A bio-feedback cup with bio-signal acquisition and feedback capability, comprising:
a cup body;
a grip being connected to the cup body;
a first electrode being located at one of the grip or the cup body;
a second electrode being located at the cup body;
a human-body temperature sensor module being located at one of the grip or the cup body;
a photoplethysmography (PPG) sensor module being located at one of the grip or the cup body;
a bio-signal acquisition module being electrically connected to the first electrode, the second electrode, a ground electrode, the human-body temperature sensor module and the PPG sensor module, and being configured to detect a user's cardiac signal with a built-in electrocardiogram (ECG or EKG) acquisition module, wherein the bio-signal acquisition module detects and obtains an ECG signal when the user touches the first electrode and the second electrode with both hands, wherein the bio-signal acquisition module obtains a PPG signal through the PPG sensor module when the user touches the PPG sensor module, and wherein the bio-signal acquisition module obtains a human-body temperature signal through the human-body temperature sensor module when the user touches the human-body temperature sensor module; and
a bio-feedback module being electrically connected to the bio-signal acquisition module and being configured to instantly give the user corresponding bio-feedback by changing a pattern on a surface of the cup body, according to a physiological index and an extended cardiac index of the user.

2. The bio-feedback cup with bio-signal acquisition and feedback capability according to claim 1, wherein the physiological index that can be used as instant feedback to the user and can be recorded, includes any one or more of human-body temperature, heart rate (beats per minute, BPM), heart rate variability (HRV), peripheral oxygen saturation (SpO2), heart age, blood pressure estimates and risk alert for excessive heart rate.

3. The bio-feedback cup with bio-signal acquisition and feedback capability according to claim 2, wherein the extended cardiac index that can be determined by a preset algorithm according to physiological data, ECG data and PPG data of the user, is a mood index, a stress index, a caffeine intake index or a heart risk index.

4. The bio-feedback cup with bio-signal acquisition and feedback capability according to claim 1, wherein the first electrode, the second electrode and the ground electrode each is a thin metal plating layer, a thin metal coating layer, a thin metal sputter coating layer or a sheet-metal layer on the surface of the cup body or the grip.

5. The bio-feedback cup with bio-signal acquisition and feedback capability according to claim 1, wherein the bio-feedback module is configured to instantly give the user corresponding bio-feedback by changing a color, a shape, a text, a carving or a background illuminating color of the pattern on the surface of the cup body, according to the physiological index and the extended cardiac index.

6. The bio-feedback cup with bio-signal acquisition and feedback capability according to claim 1, wherein the bio-feedback module is configured to instantly give the user corresponding bio-feedback by further changing a color and a displayed length of a quantifiable color indicator exposed from the cup body, according to the physiological index and the extended cardiac index.

7. The bio-feedback cup with bio-signal acquisition and feedback capability according to claim 1, further comprising a wireless data transmission module and a wireless data transmission antenna, wherein the wireless data transmission module and the wireless data transmission antenna are electrically connected to the bio-signal acquisition module for receiving and utilizing any one of Bluetooth, Wi-Fi and infrared (IR) wireless transmission technologies to transmit physiological data of the user to an electronic device.

8. The bio-feedback cup with bio-signal acquisition and feedback capability according to claim 7, wherein the wireless data transmission antenna is a thin metal plating layer, a thin metal coating layer, a thin metal sputter coating layer or a sheet-metal layer on the surface of the cup body or the grip.

9. The bio-feedback cup with bio-signal acquisition and feedback capability according to claim 7, wherein the electronic device is a mobile phone, a wearable device, a laptop or desktop computer, or a tablet computer.

10. The bio-feedback cup with bio-signal acquisition and feedback capability according to claim 9, wherein physiological data of the user is accumulated to form a historical database, stored in the electronic device, and presented by a software application of the electronic device that serves as a platform for cardio-health management and an interface for interacting with the user.

11. The bio-feedback cup with bio-signal acquisition and feedback capability according to claim 1, further comprising a power module, wherein the power module is electrically connected to the bio-signal acquisition module and is configured to provide electric power.

12. The bio-feedback cup with bio-signal acquisition and feedback capability according to claim 11, wherein the power module is a replaceable battery module, a rechargeable battery module, a detachable battery module, or a wireless rechargeable battery module.
